(19) United States
(12) Patent Application Publication
(10) Pub. No.: US 2020/0054411 A1
(43) Pub. Date: Feb. 20, 2020

(54) SURGICAL MICROSCOPE APPARATUS

(71) Applicant: MITAKA KOHKI CO., LTD., Tokyo (JP)

(72) Inventors: Katsuyuki NAKAMURA, Tokyo (JP); Masao DOI, Tokyo (JP)

(73) Assignee: MITAKA KOHKI CO., LTD., Tokyo (JP)

(21) Appl. No.: 16/540,274

(22) Filed: Aug. 14, 2019

(30) Foreign Application Priority Data
Aug. 20, 2018 (JP) 2018-153827
Mar. 22, 2019 (JP) 2019-054124

(51) Int. Cl. A61B 90/25

(52) U.S. Cl. CPC A61B 90/25; F16D 27/02

(57) ABSTRACT

A surgical microscope apparatus includes a stand base to which a stand post is fixed. The stand post supports a stand body so that the stand body horizontally turns around the stand post without turning the stand post. The stand body, therefore, is turnable with small force. Load applied to a rotary part at a top part of the stand post is only the weight of the stand body, and therefore, a bearing of the rotary part can be downsized.

### BACKGROUND OF THE INVENTION

### 1. Field of the Invention

The present invention relates to a surgical microscope apparatus.

### 2. Description of Related Art

When a doctor of, for example, neurosurgery conducts surgery by observing an enlarged view of a surgical field, the doctor uses a surgical microscope supported with a stand. The stand includes a stand base set on a floor, a stand post uprightly installed at the center of the stand base, a stand body attached to the stand post, a vertical arm rotatably attached to the stand body, and a horizontal arm horizontally extended from a top end of the vertical arm. The surgical microscope is supported at a front end of the horizontal arm.

In the stand, the stand post and stand body are integrally structured and a bottom end of the stand post is rotatably attached to the stand base. A structure above the stand base is entirely integral with the stand post and is configured to horizontally turn together with the stand post around a rotation axis of the stand post. A related art is disclosed in, for example, Japanese Unexamined Patent Application Publication No. 2016-101320 (Patent Literature 1).

According to such a related art, the stand post and stand body are integral with each other and together turn relative to the stand base, and therefore, a turning operation of the stand body requires large force. In addition, the bottom end of the stand post serves as a rotary part to which the weight of the stand post and stand body is applied, and therefore, the rotary part must have a large bearing structure to increase the weight of the stand as a whole.

### SUMMARY OF THE INVENTION

The present invention provides a surgical microscope apparatus having a stand body that is rotatable with smaller force to reduce the size of a bearing structure of a rotary part for rotating the stand body.

According to a first aspect of the present invention, the surgical microscope apparatus includes a stand base set on a floor, a stand post uprightly installed at the center of the stand base, a stand body supported at a top part of the stand post, a vertical arm having an intermediate part attached to a side face of the stand body through a horizontal shaft so that the vertical arm may turn around the horizontal shaft, and a horizontal arm horizontally extending from a top end of the vertical arm and having a front end supporting a surgical microscope, wherein the stand post is integrally fixed to the stand base in a not-rotatable manner and the stand body is supported by the stand post in such a way as to turn in a horizontal direction with respect to the stand post.

According to a second aspect of the present invention, the vertical arm and horizontal arm are formed in a balancing structure that cancels the weight of the surgical microscope with a counterweight.

### BRIEF DESCRIPTION OF THE DRAWINGS

FIG. 1 is a perspective view illustrating a surgical microscope apparatus according to an embodiment of the present invention;

FIG. 2 is an exploded side view illustrating the surgical microscope apparatus;

FIG. 3 is a front view illustrating the surgical microscope apparatus; and

FIG. 4 is a sectional view taken along a line SA-SA of FIG. 1.

### DESCRIPTION OF PREFERRED EMBODIMENTS

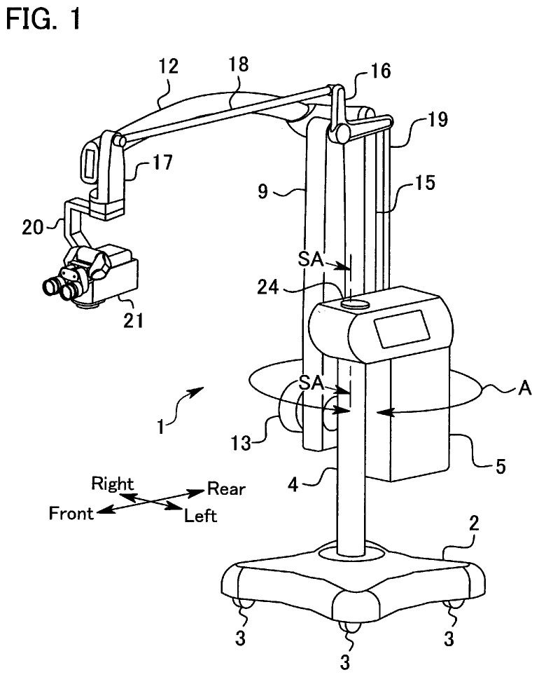

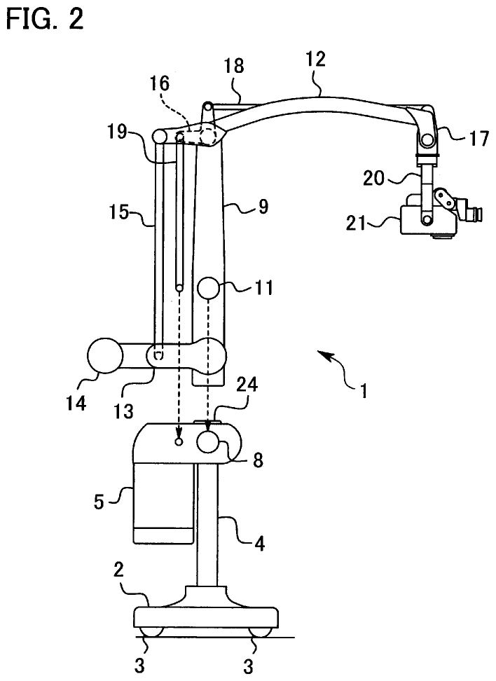

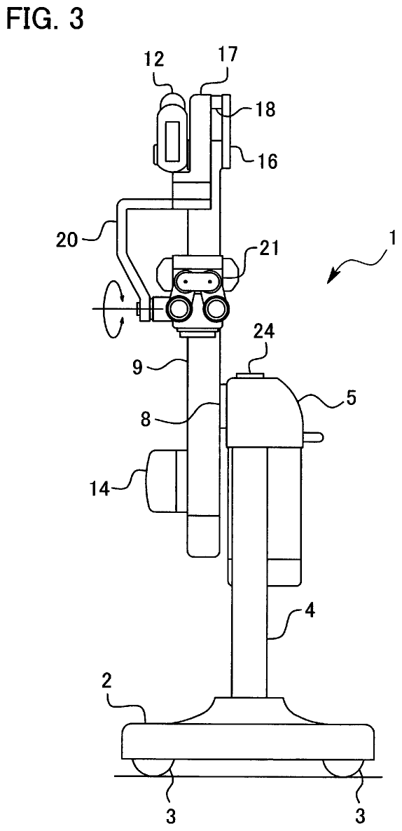

FIGS. 1 to 4 are views illustrating a surgical microscope apparatus according to an embodiment of the present invention.

The surgical microscope apparatus has a stand 1 having a stand base 2 that is set on the floor of an operation room. The stand base 2 has at each of four corners a caster 3 so that, in an unlocked state, the stand 1 is movable along the floor, and in a locked state, is fixed at a required position on the floor.

A stand post 4 is fixed with respect to the center of the stand base 2. The stand post 4 is integral with the stand base 2 and is not rotatable relative to the stand base 2.

To an upper part of the stand post 4, a stand body 5 is attached. Between the upper part of the stand post 4 and an upper part of the stand body 5, a bearing 7 is arranged so that the stand body 5 may turn in a horizontal direction (a direction A as depicted in FIG. 1) with respect to the stand post 4. Load applied to the bearing 7 at the upper part of the stand post 4 is only the weight of the stand body 5, and therefore, the bearing 7 is not necessary to be a large one. Namely, even a small bearing serves sufficiently as the bearing 7, to reduce the weight of the surgical microscope apparatus as a whole.

Between the stand post 4 and the stand body 5, an electromagnetic clutch (not illustrated) is arranged so that the stand body 5 may turn around the stand post 4 and stop at a desired turned position. A top face of the stand body 5 is provided with a cap 24 to cover a top end of the stand post 4.

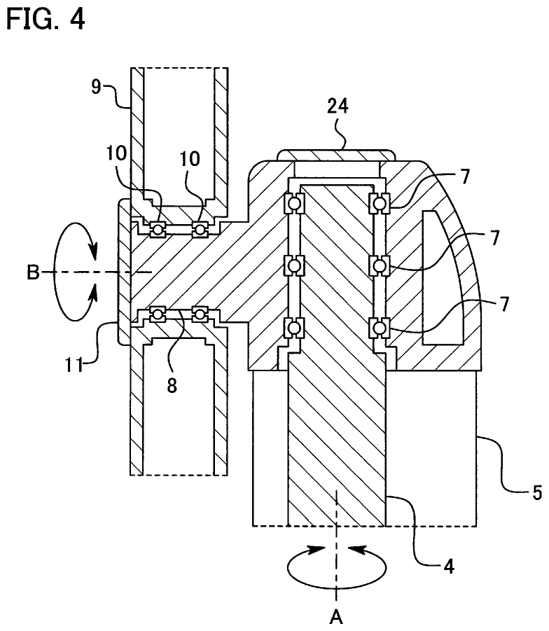

A part of the stand body 5 where the stand body 5 is rotatably supported by the stand post 4 is integrally provided with a horizontal shaft 8. The horizontal shaft 8 horizontally protrudes from a side face of the stand body 5 and rotatably supports an intermediate part of a vertical arm 9 through a bearing 10, so that the vertical arm 9 may turn in a front-rear direction as indicated with arrows in FIG. 4. A front end of the horizontal shaft 8 is covered with a cap 11 arranged on the vertical arm 9. An axis B of the rotary shaft 8 around which the vertical arm 9 attached to the stand body 5 rotates and an axis A of the stand post 4 around which the stand body 5 rotates orthogonally cross each other and are close to each other. A movable structure including the vertical arm 9 is positioned opposite to the stand body 5 with respect to the stand post 4.

A top end of the vertical arm 9 rotatably supports through a shaft a base end of a horizontal arm 12. The horizontal arm 12 is curved and extended in a horizontal direction. A lower part of the vertical arm 9 has a lower arm 13 extending in a rear direction. An end of the lower arm 13 is provided with a counterweight 14. An extended end of the horizontal arm 12 away from the vertical arm 9 is connected to a vertical sub-arm 15. The vertical sub-arm 15 vertically extends in parallel with the vertical arm 9 and a bottom end thereof is connected to an intermediate part of the lower arm 13. The vertical arm 9 and vertical sub-arm 15 form part of a vertical parallel linkage.

The top end of the vertical arm 9 supports through a shaft an L-shaped crank 16. A front end of the horizontal arm 12 supports through a shaft a lower part of a front arm 17. A top end (first end) of the crank 16 and a top end of the front arm 17 are connected to each other through a horizontal sub-arm 18 that is structurally parallel to the horizontal arm 12. The horizontal arm 12 and horizontal sub-arm 18 form part of a horizontal parallel linkage. A second end of the crank 16 is connected to a reference arm 19. The reference arm 19 vertically extends in parallel with the vertical sub-arm 15 and is connected to the stand body 5 so that the crank 16 may not rotate even when the vertical arm 9 rotates . As a result, the front arm 17, which is connected to the top end of the crank 16 through the horizontal sub-arm 18, is also not rotatable and is always maintained in a vertical state.

A lower part of the front arm 17 supports an auxiliary arm 20 that supports a surgical microscope 21. The auxiliary arm 20 is supported such that it may horizontally rotate together with the surgical microscope 21 around the front arm 17. The surgical microscope 21 is supported at a bottom end of the auxiliary arm 20 in such a way as to turn around a horizontal axis fixed at the bottom end thereof.

When a doctor on the surgical microscope apparatus wants to change an observing direction of the surgical microscope 21, the doctor may turn the stand body 5 together with the surgical microscope 21 in a horizontal direction (the direction A of FIG. 1). At this time, the stand body 5 turns without turning the standpost 4. Turning the stand body 5 without turning the stand post 4 requires only smaller force with respect to the related art. A rotary shaft (the bearing 7) for the stand body 5 is at the top part of the stand post 4, and therefore, an acting point of rotation in the direction A is in the vicinity of the bearing 7. This results in suppressing stress acting on the bearing 7 compared to the related art whose rotary shaft is very low at the stand base, thereby reducing load on the bearing 7 and stabilizing the turning operation.

In addition, a front-rear turning operation of the vertical arm 9 around the horizontal shaft 8 is also stabilized. This is because the vertical arm 9 and horizontal arm 12 form a balancing structure that cancels the weight of the surgical microscope 21 with the use of the counterweight 14. The gravity center of the structure that turns around the horizontal shaft 8 is on the horizontal shaft 8, and therefore, the vertical arm 9 is stably able to turn around the horizontal shaft 8 of the stand body 5.

As mentioned above, the horizontal shaft 8 is substantially positioned at the height of the bearing 7 arranged at the top part of the stand post 4. At the same time, the gravity center of the movable structure (including the vertical arm 9, horizontal arm 12, surgical microscope 21, and counterweight 14) that is supported to turn in the front-rear direction of the stand body 5 is positioned in the vicinity of the horizontal shaft 8. These configurations make it easy to adjust a weight balance with respect to the rotary shaft (bearing 7).

In the above explanation, the surgical microscope 21 is of an optical type having eyepieces. This is only an example. The surgical microscope 21 may be of a camera type having no eyepieces.

In summary, a surgical microscope apparatus according to a technical aspect of the present invention rotatably supports a stand body (including a surgical microscope) at a top part of a stand post fixed to a stand base so that the stand body may turn in a horizontal direction around the stand post. When turning the stand body, there is no need of turning the stand post, and therefore, the stand body is turnable with small force. Load acting on a rotary part at the top part of the stand post is only the weight of the stand body, and therefore, a bearing structure of the rotary part may be of small size to reduce the weight of the surgical microscope apparatus.

According to another technical aspect of the present invention, vertical and horizontal arms employed by the surgical microscope apparatus form a balancing structure that uses a counterweight to cancel the weight of the surgical microscope. This stabilizes rotation of the vertical arm around a horizontal shaft of the stand body.

This application claims benefit of priority under 35 USC §119 to Japanese Patent Applications No. 2018-153827 filed on Aug. 20, 2018 and No. 2019-054124 filed on Mar. 22, 2019, the entire contents of which are incorporated by reference herein.

What is claimed is:

1. A surgical microscope apparatus comprising:
a stand base set on a floor;
a stand post uprightly installed at the center of the stand base;
a stand body supported at a top part of the stand post;
a vertical arm having an intermediate part attached to a side face of the stand body through a horizontal shaft so that the vertical arm is able to turn around the horizontal shaft; and
a horizontal arm horizontally extending from a top end of the vertical arm and having a front end supporting a surgical microscope, wherein:
the stand post is integrally fixed to the stand base in a not-rotatable manner; and
the stand body is supported by the stand post in such a way as to turn in a horizontal direction with respect to the stand post.

2. The surgical microscope apparatus of claim 1, wherein the vertical arm and horizontal arm form a balancing structure that cancels the weight of the surgical microscope with the use of a counterweight.
